(19) United States
(12) Patent Application Publication
(10) Pub. No.: US 2024/0415465 A1
(43) Pub. Date: Dec. 19, 2024

(54) NOCICEPTION PREDICTION AND DETECTION USING CUMULATIVE SUM ALGORITHM AND MACHINE LEARNING CLASSIFICATION

(71) Applicant: EDWARDS LIFESCIENCES CORPORATION, Irvine, CA (US)

(72) Inventors: Christine Lee, Long Beach, CA (US); Kevin James Moses, Huntington Beach, CA (US); Cristhian M. Potes Blandon, Rancho Santa Margarita, CA (US); Feras Al Hatib, Irvine, CA (US)

(21) Appl. No.: 18/818,159

(22) Filed: Aug. 28, 2024

Related U.S. Application Data
(63) Continuation of application No. PCT/US2023/013831, filed on Feb. 24, 2023.
(60) Provisional application No. 63/314,978, filed on Feb. 28, 2022.

(51) Int. Cl. A61B 5/00; A61B 5/02; A61B 5/0205; A61B 5/021; A61B 5/024; A61B 5/08; G16H 10/60; G16H 50/30

(52) U.S. Cl. CPC A61B 5/7267; A61B 5/02028; A61B 5/02108; A61B 5/4035; A61B 5/6826; A61B 5/6852; A61B 5/7239; A61B 5/7275; A61B 5/7282; A61B 5/743; A61B 5/746; G16H 10/60; G16H 50/30; A61B 5/02007; A61B 5/0205; A61B 5/02405; A61B 5/0816; A61B 2560/0462

(57) ABSTRACT

A method is disclosed for monitoring arterial pressure of a patient and identifying nociception of the patient. The method includes receiving, by a hemodynamic monitor, sensed hemodynamic data representative of an arterial pressure waveform of the patient. Waveform analysis is performed by the hemodynamic monitor of the sensed hemodynamic data to calculate a first signal measure and a second signal measure. Both the first and second signal measures are processed by the hemodynamic monitor through a cumulative sum (CUSUM) algorithm to acquire a first CUSUM output for the first signal measure and a second CUSUM output for the second signal measure. A nociception event of the patient is detected when a change in the first CUSUM output overlaps in time with a change in the second CUSUM output. A sensory signal is outputted to a user interface of the hemodynamic monitor to warn medical personnel of the nociception event.

### CROSS-REFERENCE TO RELATED APPLICATION(S)

This application claims the benefit of International Application No. PCT/US2023/013831, filed Feb. 24, 2023, and entitled “NOCICEPTION PREDICTION AND DETECTION USING CUMULATIVE SUM ALGORITHM AND MACHINE LEARNING CLASSIFICATION,” the disclosure of which is hereby incorporated by reference in its entirety. International Application No. PCT/US2023/013831 claims the benefit of U.S. Provisional Application No. 63/314,978, filed Feb. 28, 2022, and entitled “NOCICEPTION PREDICTION AND DETECTION USING CUMULATIVE SUM ALGORITHM AND MACHINE LEARNING CLASSIFICATION,” the disclosure of which is hereby incorporated by reference in its entirety.

### BACKGROUND

The present disclosure relates generally to hemodynamic monitoring, and in particular, to detecting nociception in a patient using monitored hemodynamic data.

Nociception is the process in which nerve endings called nociceptors detect noxious stimuli and send a signal to the central nervous system, which is interpreted as pain. Noxious insult initiates a “sharp” signal from the source of the insult. Locally, nociception initiates an inflammation response. The signal then travels through neurons to the spinal column where a muscle reflex is triggered. The signal continues to the brain, where, upon reaching the lower brain, the nociception signal triggers a sympathetic nervous system response. Nociception can cause a sympathetic nervous system response without reaching consciousness or before reaching consciousness; thus, an unconscious patient in surgery or in intensive care can experience pain. To prevent a patient from awaking out of surgery or intensive care in pain, medical workers administer analgesics to the patient before and/or during surgery and at various times in the intensive care. However, knowing the amount of analgesic to administer can be difficult as pain thresholds and tolerances vary from patient to patient, and the patient is unable to verbally communicate or signal feedback while unconscious. Administering too little analgesic to the patient during surgery can result in the patient awaking in pain after the surgery. Administering too much analgesic to the patient during surgery can result in the patient experiencing nausea, drowsiness, impaired thinking skills, and impaired function.

In view of the negative consequences of administering too little analgesic to the patient and the negative consequences of administering too much analgesic to the patient, a solution is needed that will allow medical workers the ability to detect or predict nociception of an unconscious patient during surgery. Accurately detecting nociception of a patient during surgery can help medical workers know the appropriate amount of analgesic to administer to the patient so that the patient does not awake from surgery with significant pain, without providing too much analgesic to the patient.

### SUMMARY

In one example, a method is disclosed for monitoring arterial pressure of a patient and identifying nociception of the patient. The method includes receiving, by a hemodynamic monitor, sensed hemodynamic data representative of an arterial pressure waveform of the patient. The hemodynamic monitor performs waveform analysis of the sensed hemodynamic data to calculate a plurality of signal measures of the sensed hemodynamic data. Input features are extracted by the hemodynamic monitor from the plurality of signal measures that are indicative of a nociception event of the patient and a stable episode of the patient. A change detection algorithm of the hemodynamic monitor monitors at least two signal measures of the plurality of signal measures for a simultaneous change in the at least two signal measures. The hemodynamic monitor determines a nociception score representing a probability of the nociception event of the patient based on the input features. The hemodynamic monitor adjusts or maintains the nociception score based on an output of the change detection algorithm. An adjusted nociception score is displayed by the hemodynamic monitor.

In another example, a system is disclosed for monitoring arterial pressure of a patient and providing a warning to medical personnel of nociception of the patient. The system includes a hemodynamic sensor that produces hemodynamic data representative of an arterial pressure waveform of the patient. The system further includes system memory that stores nociception detection software code. A user interface includes a sensory alarm that provides a sensory signal to warn the medical personnel of a nociception event of the patient. A hardware processor is configured to execute the nociception detection software code to perform waveform analysis of the hemodynamic data to determine a plurality of signal measures. The hardware processor is further configured to execute the nociception detection software code to extract detection input features from the plurality of signal measures that are indicative of the nociception event of the patient. The hardware processor is further configured to execute the nociception detection software code to monitor at least two signal measures from the plurality of signal measures for a simultaneous change in the at least two signal measures. The hardware processor also is configured to execute the nociception detection software code to determine, based on the detection input features and a presence of the simultaneous change in the at least two signal measures, a nociception score representing a probability of the nociception event of the patient. The sensory alarm of the user interface is invoked in response to the nociception score satisfying a predetermined detection criterion.

In another example, a method is disclosed for monitoring arterial pressure of a patient and identifying nociception of the patient. The method includes receiving, by a hemodynamic monitor, sensed hemodynamic data representative of an arterial pressure waveform of the patient. Waveform analysis is performed by the hemodynamic monitor of the sensed hemodynamic data to calculate a first signal measure and a second signal measure of the sensed hemodynamic data. Both the first signal measure and the second signal measure are processed by the hemodynamic monitor through a cumulative sum (CUSUM) algorithm to acquire a first CUSUM output for the first signal measure and a second CUSUM output for the second signal measure. The first CUSUM output and the second CUSUM output are monitored by the hemodynamic monitor for a change in both the first CUSUM output and the second CUSUM output. A nociception event of the patient is detected when the change in the first CUSUM output overlaps in time with the change in the second CUSUM output. A sensory signal is outputted to a user interface of the hemodynamic monitor to warn medical personnel of the nociception event of the patient.

In another example, a system is disclosed for monitoring arterial pressure of a patient and providing a warning to medical personnel of nociception of the patient. The system includes a hemodynamic sensor that produces hemodynamic data representative of an arterial pressure waveform of the patient. Nociception detection software code is stored in a system memory. The system further includes a user interface that includes a sensory alarm that provides a sensory signal to warn the medical personnel of a nociception event of the patient. The system also includes a hardware processor that is configured to execute the nociception detection software code to perform waveform analysis of the hemodynamic data to determine a first signal measure and a second signal measure. Both the first signal measure and the second signal measure are processed through a cumulative sum (CUSUM) algorithm to acquire a first CUSUM output for the first signal measure and a second CUSUM output for the second signal measure. The first CUSUM output and the second CUSUM output are monitored over time for a change in both the first CUSUM output and the second CUSUM output. A nociception event of the patient is detected when the change in the first CUSUM output overlaps in time with the change in the second CUSUM output. A sensory signal is outputted to a user interface of the hemodynamic monitor to warn medical personnel of the nociception event of the patient.

### BRIEF DESCRIPTION OF THE DRAWINGS

FIG. 1 is a perspective view of an example hemodynamic monitor that analyzes an arterial pressure of a patient and provides a risk score and a warning to medical personnel of a nociception event of the patient.

FIG. 2 is a perspective view of an example minimally invasive pressure sensor for sensing hemodynamic data representative of arterial pressure of a patient.

FIG. 3 is a perspective view of an example non-invasive sensor for sensing hemodynamic data representative of arterial pressure of a patient.

FIG. 4 is a block diagram illustrating an example hemodynamic monitoring system that determines risk scores representing a probability of a current nociception event, a future nociception event, a current hemodynamic drug administration event, a future hemodynamic drug administration event, and/or a stable period for a patient based on a set of input features derived from signal measures of an arterial pressure waveform of the patient.

FIG. 5 is a diagram of a clinical dataset and with clinical annotations used for machine training of the hemodynamic monitoring system.

FIG. 6 is a graph illustrating a systolic blood pressure and a heart rate over time from the clinical dataset of FIG. 5 and showing a nociception event and an administration of an analgesic.

FIG. 7 is a graph illustrating a systolic blood pressure and a heart rate over time from the clinical dataset of FIG. 5 and showing a stable episode.

FIG. 8 is a graph illustrating a systolic blood pressure and a heart rate over time from the clinical dataset of FIG. 5 and showing a hemodynamic drug administration event and an administration of a vasopressor drug.

FIG. 9 is a flow diagram for extracting a set of input features derived from waveform features of an arterial pressure waveform of a patient for training a machine learning model of a hemodynamic monitoring system.

FIG. 10 is a graph illustrating an example trace of an arterial pressure waveform including example indicia corresponding to signal measures used to extract the input features that determine the risks scores of the patient.

FIG. 11 is a graph illustrating a systolic blood pressure, a heart rate, and an algorithmic output for the systolic blood pressure and heart rate all plotted over time.

FIG. 12 is a series of graphs plotted over time illustrating how the algorithmic output for the systolic blood pressure and/or the heart rate is generated by a CUSUM algorithm.

FIG. 13 is a flow chart illustrating a method of refining nociception prediction and detection outputs of a machine learning model.

FIG. 14 is a graph illustrating machine learning and algorithmic outputs for systolic blood pressure, heart rate, and probability of nociception.

### DETAILED DESCRIPTION

As described herein, a hemodynamic monitoring system implements a predictive model that calculates a risk score representing a probability of a current nociception event for a patient. The model's risk score can be refined using a change detection algorithm to analyze selected hemodynamic parameters most closely correlated with nociception events.

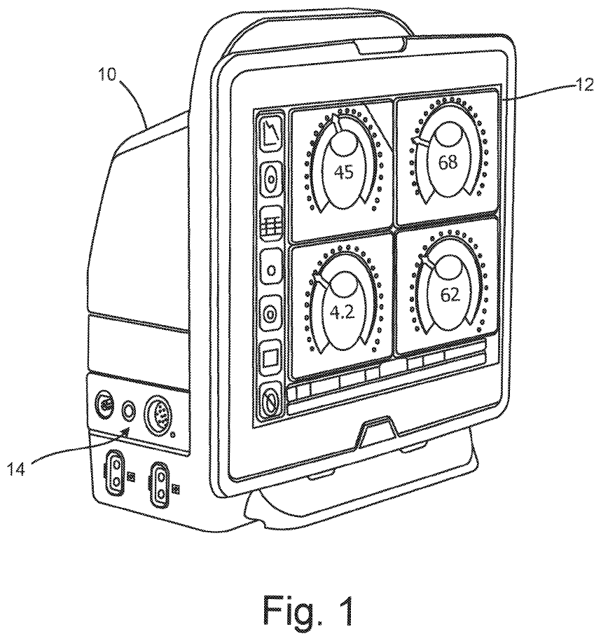

The machine learning of the predictive models of the hemodynamic monitoring system are trained using a clinical dataset containing arterial pressure waveforms labeled with clinical annotations of administration of analgesics, vasopressors, inotropes, fluids, and other medication that alter cardiovascular hemodynamics. The hemodynamic monitoring system is described in detail below with reference to FIGS. 1-13.

FIG. 1 is a perspective view of hemodynamic monitor 10 that determines a score representing a probability of a current nociception event of a patient. As illustrated in FIG. 1, hemodynamic monitor 10 includes display 12 that, in the example of FIG. 1, presents a graphical user interface including control elements (e.g., graphical control elements) that enable user interaction with hemodynamic monitor 10. Hemodynamic monitor 10 can also include a plurality of input and/or output (I/O) connectors configured for wired connection (e.g., electrical and/or communicative connection) with one or more peripheral components, such as one or more hemodynamic sensors, as is further described below. For instance, as illustrated in FIG. 1, hemodynamic monitor 10 can include I/O connectors 14. While the example of FIG. 1 illustrates five separate I/O connectors 14, it should be understood that in other examples, hemodynamic monitor 10 can include fewer than five I/O connectors or greater than five I/O connectors. In yet other examples, hemodynamic monitor 10 may not include I/O connectors 14, but rather may communicate wirelessly with various peripheral devices.

As further described below, hemodynamic monitor 10 includes one or more processors and computer-readable memory that stores nociception detection and prediction software code which is executable to produce a score representing a probability of a present (i.e., current) nociception event for a patient. Hemodynamic monitor 10 can receive sensed hemodynamic data representative of an arterial pressure waveform of the patient, such as via one or more hemodynamic sensors connected to hemodynamic monitor 10 via I/O connectors 14. Hemodynamic monitor 10 executes the nociception prediction software code to obtain, using the received hemodynamic data, multiple nociception profiling parameters (e.g., input features), which can include one or more vital sign parameters characterizing vital sign data of the patient, as well as differential and combinatorial parameters derived from the one or more vital sign parameters, as is further described below.

As illustrated in FIG. 1, hemodynamic monitor 10 can present a graphical user interface at display 12. Display 12 can be a liquid crystal display (LCD), a light-emitting diode (LED) display, an organic light-emitting diode (OLED) display, or other display device suitable for providing information to users in graphical form. In some examples, such as the example of FIG. 1, display 12 can be a touch-sensitive and/or presence-sensitive display device configured to receive user input in the form of gestures, such as touch gestures, scroll gestures, zoom gestures, swipe gestures, or other gesture input.

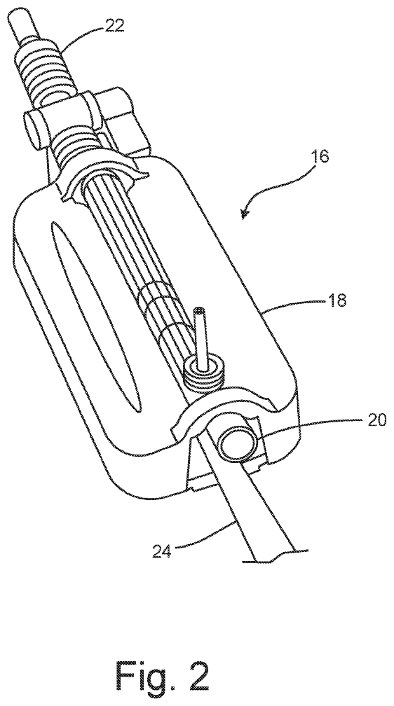

FIG. 2 is a perspective view of hemodynamic sensor 16 that can be attached to a patient for sensing hemodynamic data representative of arterial pressure of the patient. Hemodynamic sensor 16, illustrated in FIG. 2, is one example of a minimally invasive hemodynamic sensor that can be attached to the patient via, e.g., a radial arterial catheter inserted into an arm of the patient. In other examples, hemodynamic sensor 16 can be attached to the patient via a femoral arterial catheter inserted into a leg of the patient.

As illustrated in FIG. 2, hemodynamic sensor 16 includes housing 18, fluid input port 20, catheter-side fluid port 22, and I/O cable 24. Fluid input port 20 is configured to be connected via tubing or other hydraulic connection to a fluid source, such as a saline bag or other fluid input source. Catheter-side fluid port 22 is configured to be connected via tubing or other hydraulic connection to a catheter (e.g., a radial arterial catheter or a femoral arterial catheter) that is inserted into an arm of the patient (i.e., a radial arterial catheter) or a leg of the patient (i.e., a femoral arterial catheter). I/O cable 24 is configured to connect to hemodynamic monitor 10 via, e.g., one or more of I/O connectors 14 (FIG. 1). Housing 18 of hemodynamic sensor 16 encloses one or more pressure transducers, communication circuitry, processing circuitry, and corresponding electronic components to sense fluid pressure corresponding to arterial pressure of the patient that is transmitted to hemodynamic monitor 10 (FIG. 1) via I/O cable 24.

In operation, a column of fluid (e.g., saline solution) is introduced from a fluid source (e.g., a saline bag) through hemodynamic sensor 16 via fluid input port 20 to catheter-side fluid port 22 toward the catheter inserted into the patient. Arterial pressure is communicated through the fluid column to pressure sensors located within housing 16 which sense the pressure of the fluid column. Hemodynamic sensor 16 translates the sensed pressure of the fluid column to an electrical signal via the pressure transducers and outputs the corresponding electrical signal to hemodynamic monitor 10 (FIG. 1) via I/O cable 24. Hemodynamic sensor 16 therefore transmits analog sensor data (or a digital representation of the analog sensor data) to hemodynamic monitor 10 (FIG. 1) that is representative of substantially continuous beat-to-beat monitoring of the arterial pressure of the patient.

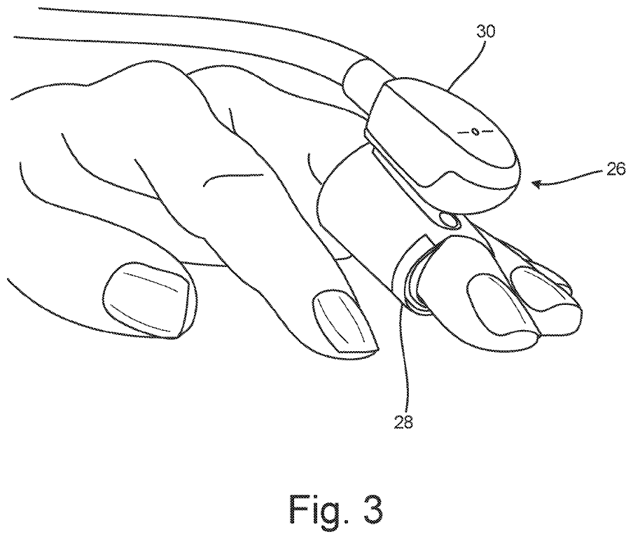

FIG. 3 is a perspective view of hemodynamic sensor 26 for sensing hemodynamic data representative of arterial pressure of a patient. Hemodynamic sensor 26, illustrated in FIG. 3, is one example of a non-invasive hemodynamic sensor that can be attached to the patient via one or more finger cuffs to sense data representative of arterial pressure of the patient. As illustrated in FIG. 3, hemodynamic sensor 26 includes inflatable finger cuff 28 and heart reference sensor 30. Inflatable finger cuff 28 includes an inflatable blood pressure bladder configured to inflate and deflate as controlled by a pressure controller (not illustrated) that is pneumatically connected to inflatable finger cuff 28. Inflatable finger cuff 28 also includes an optical (e.g., infrared) transmitter and an optical receiver that are electrically connected to the pressure controller (not illustrated) to measure the changing volume of the arteries under the cuff in the finger.

In operation, the pressure controller continually adjusts pressure within the finger cuff to maintain a constant volume of the arteries in the finger (i.e., the unloaded volume of the arteries) as measured via the optical transmitter and optical receiver of inflatable finger cuff 28. The pressure applied by the pressure controller to continuously maintain the unloaded volume is representative of the blood pressure in the finger, and is communicated by the pressure controller to hemodynamic monitor 10 shown in FIG. 1. Heart reference sensor 30 measures the hydrostatic height difference between the level at which the finger is kept and the reference level for the pressure measurement, which typically is heart level. Accordingly, hemodynamic sensor 26 transmits sensor data that is representative of substantially continuous beat-to-beat monitoring of the arterial pressure waveform of the patient.

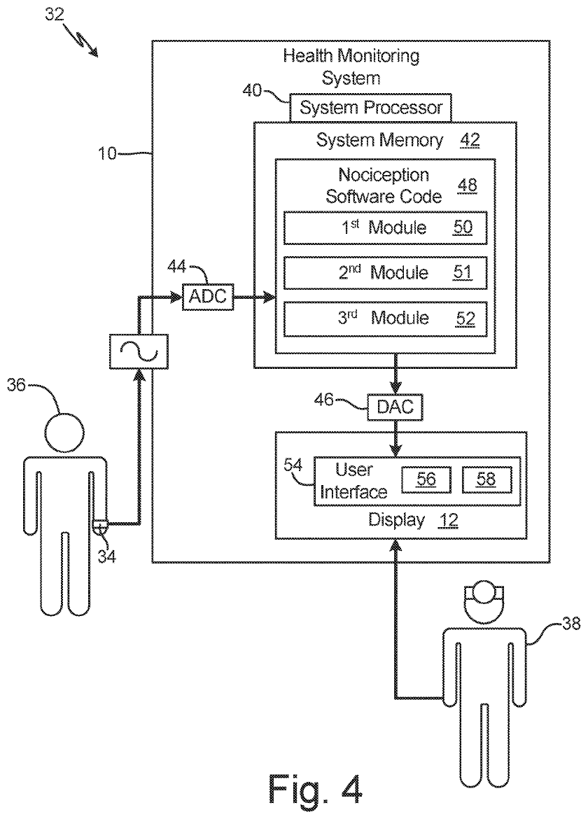

As illustrated in FIG. 4, system memory 42 stores nociception software code 48 which forms the predictive model of hemodynamic monitor 10. Nociception software code 48 includes first module 50 for extracting and calculating waveform features from the arterial pressure of patient 36, second module 51 for extracting input features from the waveform features, and third module 52 for calculating probability of nociception of patient 36 based on the input features. Display 12 provides user interface 54, which includes control elements 56 that enable user interaction with hemodynamic monitor 10 and/or other components of hemodynamic monitoring system 32. User interface 54, as illustrated in FIG. 4, also provides sensory alarm 58 to provide warning to medical personnel of a current nociception event of patient 36, as is further described below. Sensory alarm 58 can be implemented as one or more of a visual alarm, an audible alarm, a haptic alarm, or other type of sensory alarm. For instance, sensory alarm 58 can be invoked as any combination of flashing and/or colored graphics shown by user interface 54 on display 12, display of the nociception score via user interface 54 on display 12, a warning sound such as a siren or repeated tone, and a haptic alarm configured to cause hemodynamic monitor 10 to vibrate or otherwise deliver a physical impulse perceptible to medical worker 38 or other user.

Hemodynamic sensor 34 can be attached to patient 36 to sense hemodynamic data representative of the arterial pressure waveform of patient 36. Hemodynamic sensor 34 is operatively connected to hemodynamic monitor 10 (e.g., electrically and/or communicatively connected via wired or wireless connection, or both) to provide the sensed hemodynamic data to hemodynamic monitor 10. In some examples, hemodynamic sensor 34 provides the hemodynamic data representative of the arterial pressure waveform of patient 36 to hemodynamic monitor 10 as an analog signal, which is converted by ADC 44 to digital hemodynamic data representative of the arterial pressure waveform. In other examples, hemodynamic sensor 34 can provide the sensed hemodynamic data to hemodynamic monitor 10 in digital form, in which case hemodynamic monitor 10 may not include or utilize ADC 44. In yet other examples, hemodynamic sensor 34 can provide the hemodynamic data representative of the arterial pressure waveform of patient 36 to hemodynamic monitor 10 as an analog signal, which is analyzed in its analog form by hemodynamic monitor 10.

Hemodynamic sensor 34 can be a non-invasive or minimally invasive sensor attached to patient 36. For instance, hemodynamic sensor 34 can take the form of minimally invasive hemodynamic sensor 16 (FIG. 2), non-invasive hemodynamic sensor 26 (FIG. 3), or other minimally invasive or non-invasive hemodynamic sensor. In some examples, hemodynamic sensor 34 can be attached non-invasively at an extremity of patient 36, such as a wrist, an arm, a finger, an ankle, a to, or other extremity of patient 36. As such, hemodynamic sensor 34 can take the form of a small, lightweight, and comfortable hemodynamic sensor suitable for extended wear by patient 36 to provide substantially continuous beat-to-beat monitoring of the arterial pressure of patient 36 over an extended period of time, such as minutes or hours.

In certain examples, hemodynamic sensor 34 can be configured to sense an arterial pressure of patient 36 in a minimally invasive manner. For instance, hemodynamic sensor 34 can be attached to patient 36 via a radial arterial catheter inserted into an arm of patient 36. In other examples, hemodynamic sensor 34 can be attached to patient 36 via a femoral arterial catheter inserted into a leg of patient 36. Such minimally invasive techniques can similarly enable hemodynamic sensor 34 to provide substantially continuous beat-to-beat monitoring of the arterial pressure of patient 36 over an extended period of time, such as minutes or hours.

System processor 40 is a hardware processor configured to execute nociception software code 48, which implements first module 50, second module 51, and third module 52 to produce a nociception score representing a probability of a current nociception event for patient 36. Examples of system processor 40 can include any one or more of a microprocessor, a controller, a digital signal processor (DSP), an application specific integrated circuit (ASIC), a field-programmable gate array (FPGA), or other equivalent discrete or integrated logic circuitry.

System memory 42 can be configured to store information within hemodynamic monitor 10 during operation. System memory 42, in some examples, is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). System memory 42 can include volatile and non-volatile computer-readable memories. Examples of volatile memories can include random access memories (RAM), dynamic random access memories (DRAM), static random access memories (SRAM), and other forms of volatile memories. Examples of non-volatile memories can include, e.g., magnetic hard discs, optical discs, flash memories, or forms of electrically programmable memories (EPROM) or electrically erasable and programmable (EEPROM) memories.

Display 12 can be a liquid crystal display (LCD), a light-emitting diode (LED) display, an organic light-emitting diode (OLED) display, or other display device suitable for providing information to users in graphical form. User interface 54 can include graphical and/or physical control elements that enable user input to interact with hemodynamic monitor 10 and/or other components of hemodynamic monitoring system 32. In some examples, user interface 54 can take the form of a graphical user interface (GUI) that presents graphical control elements presented at, e.g., a touch-sensitive and/or presence sensitive display screen of display 12. In such examples, user input can be received in the form of gesture input, such as touch gestures, scroll gestures, zoom gestures, or other gesture input. In certain examples, user interface 54 can take the form of and/or include physical control elements, such as a physical buttons, keys, knobs, or other physical control elements configured to receive user input to interact with components of hemodynamic monitoring system 32.

In operation, hemodynamic sensor 34 senses hemodynamic data representative of an arterial pressure waveform of patient 36. Hemodynamic sensor 34 provides the hemodynamic data (e.g., as analog sensor data), to hemodynamic monitor 10. ADC 44 converts the analog hemodynamic data to digital hemodynamic data representative of the arterial pressure waveform of the patient.

Hemodynamic monitoring system 32 can discern when patient 36 is experiencing a current nociception event and when patient 36 is merely reacting to a hemodynamic drug previously administered to patient 36 by medical worker 38 (hereinafter referred to as a hemodynamic drug administration event). The hemodynamic drug administration event is defined as an event where patient 36 experiences an increase in heart rate and an increase in blood pressure due to the administration of a compound that alters cardiovascular hemodynamics (e.g., analgesics, vasopressors, inotropes, fluids, and/or other medication) and is not a nociception event of patient 36. Nociception software code 48 includes hemodynamic drug detection software code for detecting the presence of hemodynamic drug administration event of patient 36. System processor 40 executes the hemodynamic drug detection software code of nociception software code 48 to determine, using the received hemodynamic data, a hemodynamic drug detection score representing a probability that the hemodynamic drug administration event is responsible for increasing a heart rate and a blood pressure of patient 36. For instance, system processor 40 can execute first module 50 to perform waveform analysis of the hemodynamic data to determine the plurality of signal measures. System processor 40 executes second module 51 to extract hemodynamic drug detection input features from the plurality of signal measures that detect the current effects of the hemodynamic drug administration event of patient 36. System processor 40 executes third module 52 to determine, based on the hemodynamic drug detection input features, the hemodynamic drug detection score of patient 36. If the hemodynamic drug detection score satisfies a predetermined hemodynamic detection criterion, system processor 40 invokes sensory alarm 58 of user interface 54 to send a third sensory signal to alert medical worker 38 that patient 36 is experiencing a hemodynamic drug administration event and not a current nociception event. The hemodynamic drug detection score and the third sensory signal help to prevent medical worker 38 from confusing the hemodynamic drug administration event with a nociception event and prevent medical worker 38 from unnecessarily administering analgesics to patient 36.

System memory 42 of hemodynamic monitor 10 can also include stable detection software code for detecting a stable episode of patient 36. The stable episode is defined as a period during which patient 36 does not experience a nociception event or a hemodynamic drug administration event. The stable detection software code can be a subpart of nociception software code 48. System processor 40 executes stable detection software code to extract stable detection input features from the plurality of signal measures. Stable detection software code can extract the stable detection input features from the plurality of signal measures using second module 51. The stable detection input features detect the stable episode of patient 36. System processor 40 executes third module 52 to determine, based on the stable detection input features, a stable score of patient 36. System processor 40 outputs the stable score of patient 36 to user interface 54 of display 12.

System processor 40 can execute first module 50 to extract a single batch of the plurality of signal measures for a given unit of time, and that single batch of signal measures can be used by second module 51 to extract all of nociception detection input features, hemodynamic drug detection input features, and stable detection input features for that unit of time. Second module 51 can extract all of nociception detection input features, hemodynamic drug detection input features, and stable detection input features concurrently from the plurality of signal measures. System processor 40 can execute third module 52 to concurrently determine the nociception detection score, the hemodynamic drug detection score, and the stable score. Nociception software code 48 of hemodynamic monitor 10 can utilize, in some examples, a classification-type machine learning model with binary positive versus negative labels to label selected data segments for training of machine learning models, as is discussed in greater detail below. Processor 40 can, in certain examples, output the nociception detection score and the hemodynamic drug detection score together to display 12 to compare and contrast the two probabilities and help medical worker 38 better understand whether a nociception event or a hemodynamic drug administration event is causing the increase in blood pressure and heart rate in patient 36.

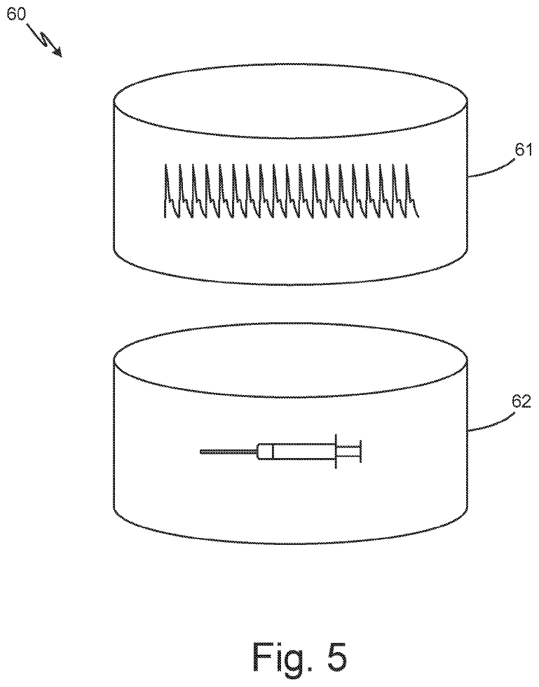

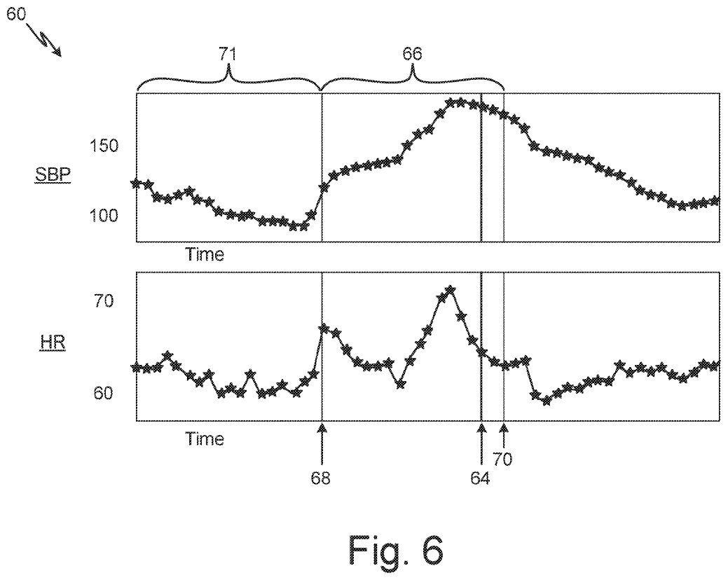

FIG. 5 is a diagram of clinical dataset 60 used for machine training of the hemodynamic monitor 10. Clinical dataset 60 includes first dataset 61 containing a collection of arterial pressure waveforms recorded from previous patients. First dataset 61 can be collected by invasive hemodynamic sensors, such as hemodynamic sensor 16 shown in FIG. 2, or collected by non-invasive hemodynamic sensors, such as hemodynamic sensor 26 shown in FIG. 3. Clinical dataset 60 also includes second dataset 62 containing a log of instances where compounds that alter cardiovascular hemodynamics (e.g., analgesics, vasopressors, inotropes, fluids, and/or other medication) were administered to the patients of first dataset 61 while their arterial pressure waveforms were being recorded. Medical workers can enter the administration information directly into the same hemodynamic monitors that are collecting first dataset 61, such that first dataset 61 and second dataset 62 are collected together concurrently. As shown in FIGS. 6-8, the information in second dataset 62 is annotated and labeled onto the collection of arterial pressure waveforms of first dataset 61.

FIG. 6 is a graph illustrating a plot of systolic blood pressure over time (hereinafter referred to as “SBP plot”) and a plot of heart rate over time (hereinafter referred to as “HR plot”). Before clinical dataset 60 can be used to machine train hemodynamic monitor 10, the SBP plot and the HR plot are determined for each of the arterial pressure waveforms collected in clinical dataset 60. The SBP plot and the HR plot shown in FIG. 6 are an example from one of the arterial pressure waveforms (not shown) in clinical dataset 60. After the SBP plot and the HR plot are determined for each of the arterial pressure wave forms collected in clinical dataset 60, the SBP plot and the HR plot are both annotated to show when compounds that alter the cardiovascular hemodynamics were administered to the clinical patient. For example, the SBP plot and the HR plot shown in FIG. 6 include analgesic label 64, which is a vertical bar extending across the SBP plot and the HR plot at the same position in time. Analgesic label 64 in FIG. 6 indicates that the clinical patient was administered an analgesic during the time represented by the SBP plot and the HR plot in FIG. 6. After the SBP plot and the HR plot are annotated and labeled to show drug administrations to the clinical patient, nociception data segments 66 are identified and labeled on the SBP plot and the HR plot.

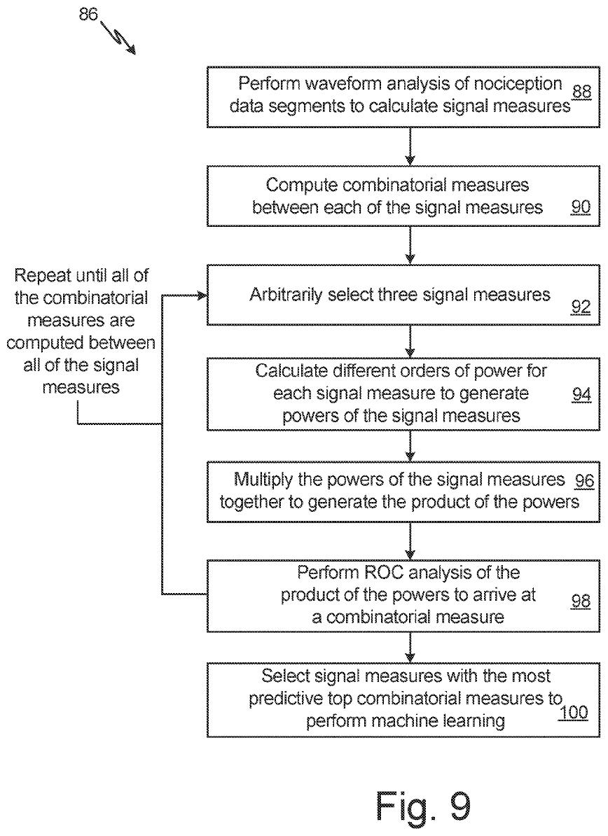

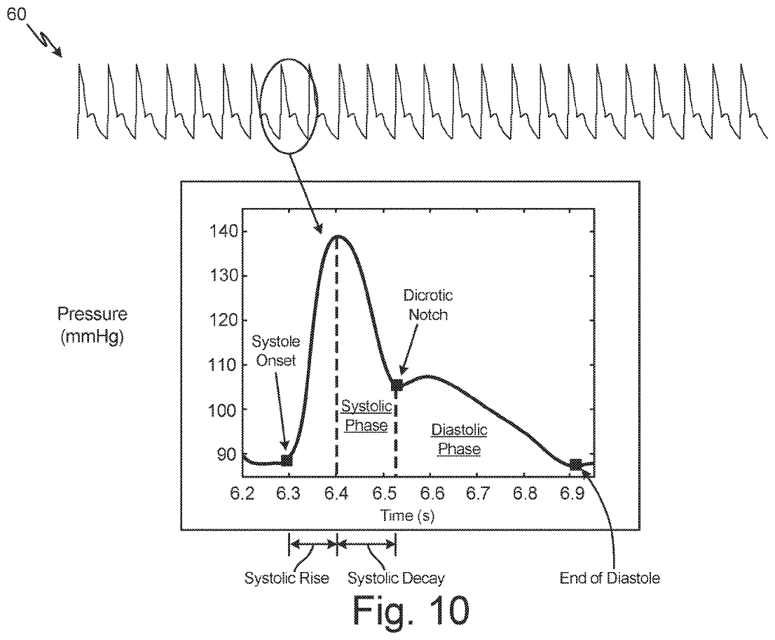

As shown in FIG. 6, nociception data segments 66 are identified on the SBP plot and the HR plot by locating time segments in both the SBP plot and the HR plot where systolic blood pressure of the clinical patient increases by at least a threshold amount (e.g., 20% or other threshold amounts) compared to a prior time period, where heart rate of the clinical patient also increases by at least a threshold amount (e.g. 20% or other threshold amounts) compared to the prior time period, and there has been no infusion of a compound that alters cardiovascular hemodynamics (e.g., analgesics, vasopressors, inotropes, fluids, and/or other medication) started prior to the increase in blood pressure and the increase in heart rate. In FIG. 6, both the SBP plot and the HR plot increase by more than 20% at starting point 68, which occurs before analgesic label 64, thus indicating the start of nociception data segment 66 in FIG. 6. Nociception data segment 66 in FIG. 6 continues until both the SBP plot and the HR plot begin to drop as a result of the analgesic administered in time to the clinical patient at analgesic label 64. The drops in the SBP plot and the HR plot are indicated by ending point 70. Starting point 68 and ending point 70 are both labeled on the HR plot and the SBP plot and the time segment between starting point 68 and ending point 70 is designated as one nociception data segment 66. With analgesic label 64 and nociception data segment 66 identified on the SBP plot and the HR plot, the arterial pressure waveform used to generate the SBP plot and the HR plot in FIG. 6 can also be annotated and labeled to show when analgesic label 64 and nociception data segment 66 occurred on the arterial pressure waveform. Once labeled with analgesic label 64 and nociception data segment 66, the arterial pressure waveform is ready to be used for machine training of the hemodynamic monitor 10 to detect nociception events. As will be discussed further below with reference to FIGS. 9-10, waveform analysis is performed on clinical dataset 60 containing nociception data segments 66 to calculate a plurality of signal measures which are then used to compute the nociception detection input features that best detect the probability of current nociception events.

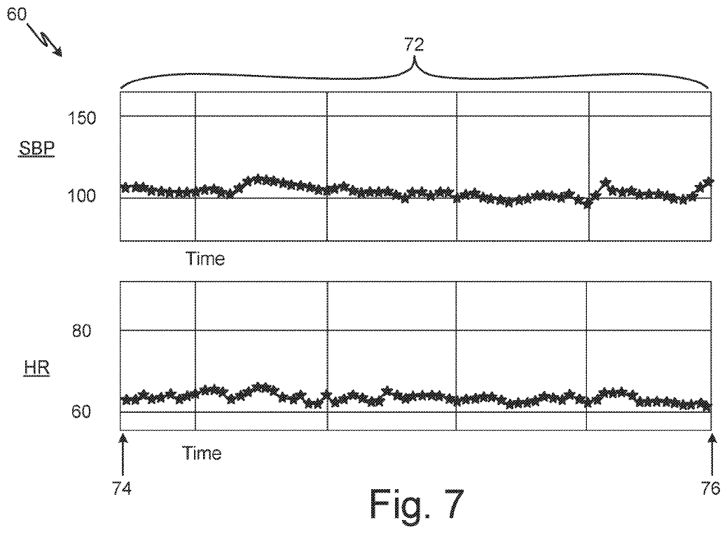

FIG. 7 is a graph illustrating another SBP plot and HR plot derived from an arterial pressure waveform segment (not shown) from clinical dataset 60. The arterial pressure waveform segment that produced the SBP plot and the HR plot in FIG. 7 can be identified as a stable data segment 72 and used for data mining and machine training hemodynamic monitor 10 to detect when a patient is experiencing a stable episode with no nociception. An arterial pressure waveform segment in clinical dataset 60 is identified as stable data segment 72 if there is no increase greater than a threshold mount (e.g. 20% or other threshold amounts) in the SBP plot, no increase greater than a threshold amount (e.g. 20% or other threshold amount) in the HR plot, and no infusion performed of a compound that alters cardiovascular hemodynamics. As shown in the example of FIG. 7, the SBP plot does not include an increase greater than 20% between starting point 74 and ending point 76. The HR plot in the example of FIG. 7 also does not include an increase greater than 20% between starting point 74 and ending point 76. The HR plot and the SBP plot of FIG. 7 also does not include any annotations or labels indicating an infusion of a compound that alters cardiovascular hemodynamics in the clinical patient between starting point 74 and ending point 76. Given the above-described characteristics of the HR plot and the SBP plot in the example of FIG. 7, the HR plot and the SBP plot of FIG. 7 are labeled as a stable data segment 72 between starting point 74 and ending point 76. The arterial pressure waveform segment (not shown) that generated the HR plot and the SBP plot of FIG. 7 is also labeled as a stable data segment 72 between starting point 74 and ending point 76. Once labeled with stable data segment 72, the arterial pressure waveform segment is ready to be used for stable machine training of the hemodynamic monitor 10. As will be discussed further below with reference to FIGS. 9-10, waveform analysis is performed on clinical dataset 60 containing stable data segments 72 to calculate a plurality of signal measures which are then used to compute the stable detection input features that best detect the probability of stable episodes.

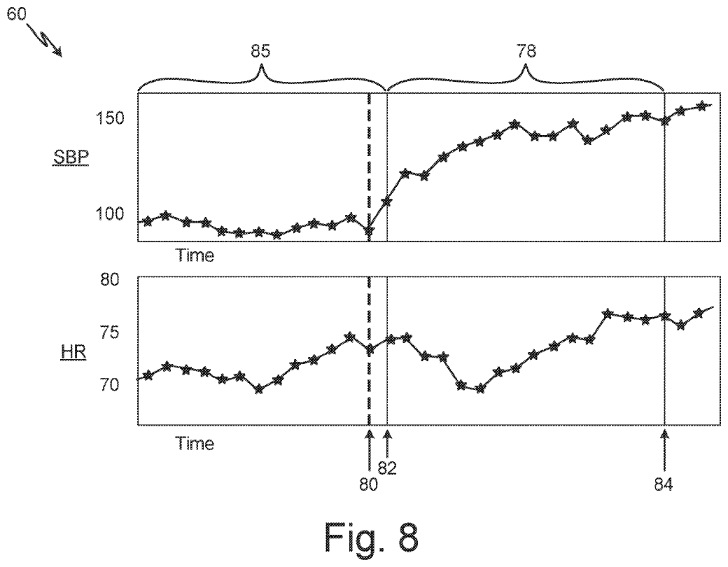

FIG. 8 is a graph illustrating another SBP plot and HR plot derived from an arterial pressure waveform segment (not shown) from clinical dataset 60. The arterial pressure waveform segment that produced the SBP plot and HR plot in FIG. 8 can be identified as a hemodynamic drug administration data segment 78 (referred to hereinafter as “HDA data segment 78”) and used for data mining and machine training hemodynamic monitor 10 to detect when a patient is experience a current hemodynamic drug administration event. An arterial pressure waveform segment in clinical dataset 60 is identified as a HDA data segment 78 if the arterial pressure waveform segment includes an infusion of a compound that alters cardiovascular hemodynamics into the clinical patient and there is an increase of at least at least a threshold amount (e.g. 20% or other threshold amounts) in both the SBP plot and the HR plot after the infusion. In the example of FIG. 8, vasopressor infusion label 80 on the SBP plot and the HR plot indicates that the clinical patient was administered a vasopressor drug. Shortly after vasopressor infusion label 80, the SBP plot increased by at least 20% between starting point 82 and ending point 84. Between starting point 82 and ending point 84, HR plot also increased by at least 20%, thus indicating that a HDA data segment 78 occurred between starting point 82 and ending point 84. The HR plot and the SBP plot of FIG. 8 are labeled as a HDA data segment 78 between starting point 82 and ending point 84. The arterial pressure waveform segment (not shown) that generated the HR plot and the SBP plot of FIG. 8 is also labeled as a HDA data segment 78 between starting point 82 and ending point 84. Once labeled with HDA data segment 78, the arterial pressure waveform segment is ready to be used for hemodynamic drug detection machine training of the hemodynamic monitor 10. As will be discussed further below with reference to FIGS. 9-10, waveform analysis is performed on clinical dataset 60 containing HDA data segments 78 to calculate a plurality of signal measures which are then used to compute the hemodynamic drug detection input features that best detect the probability of current hemodynamic drug administration events.

FIG. 9 is a flow diagram of method 86 for data mining clinical dataset 60 from FIGS. 5-8 for machine training the machine learning model of hemodynamic monitor 10. Method 86 in FIG. 9 will be discussed while also referencing FIG. 10. Method 86 is applied to each of nociception data segments 66 (shown in FIG. 6), prediction data segments 71 (shown in FIG. 6), stable data segments 72 (shown in FIG. 7), and HDA data segments 78 (shown in FIG. 8) in clinical dataset 60 to train hemodynamic monitor to find the input features previously described with reference to FIG. 4. Method 86 will be described as applied to nociception data segments 66 (shown in FIG. 6).

To machine train hemodynamic monitor 10 to identify the nociception detection input features described in FIG. 4, nociception detection input features are first determined by applying method 86 to nociception data segments 66 of clinical dataset 60. First step 88 of method 86 is to perform waveform analysis of nociception data segments 66 of the arterial waveforms collected in dataset 60 to calculate a plurality of signal measures of the nociception data segments. Performing waveform analysis of nociception data segments 66 can include identifying individual cardiac cycles in each of the arterial pressure waveforms of nociception data segments 66. FIG. 10 provides an example graph illustrating an example trace of an arterial pressure waveform with an individual cardiac cycle identified and enlarged. Next, performing waveform analysis of nociception data segments 66 can include identifying a dicrotic notch in each of the individual cardiac cycles of each of the arterial pressure waveforms of nociception data segments 66, similar to the example shown in FIG. 10. Next, the waveform analysis on nociception data segments 66 includes identifying a systolic rise phase, a systolic decay phase, and a diastolic phase in each of the individual cardiac cycles of each of the arterial pressure waveforms of nociception data segments 66, similar to the example shown in FIG. 10.

Signal measures are extracted from each of the systolic rise phase, the systolic decay phase, and the diastolic phase from each of the individual cardiac cycles of each of the arterial pressure waveforms of nociception data segments 66. The signal measures can correspond to hemodynamic effects from each of the systolic rise phase, the systolic decay phase, and the diastolic phase from each of the individual cardiac cycles. Those hemodynamic effects can include contractility, aortic compliance, stroke volume, vascular tone, afterload, and full cardiac cycle. The signal measures calculated or extracted by the waveform analysis of first step 88 of method 86 includes a mean, a maximum, a minimum, a duration, an area, a standard deviation, derivatives, and/or morphological measures from each of the systolic rise phase, the systolic decay phase, the systolic phase and the diastolic phase from each of the individual cardiac cycles. The signal measures can also include heart rate, respiratory rate, stroke volume, pulse pressure, pulse pressure variation (PPV), stroke volume variation (SVV), mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles of each of the arterial pressure waveforms of nociception data segments 66.

After the signal measures are determined for nociception data segments 66, step 90 of method 86 is performed on the signal measures of nociception data segments 66. Step 90 of method 86 is to compute combinatorial measures between each of the signal measures of nociception data segments 66. Computing the combinatorial measures between the signal measures of nociception data segments 66 can include performing steps 92, 94, 96, and 98 shown in FIG. 9 on all of the signal measures of nociception data segments 66.

Step 92 of method 86 is to arbitrarily select three signal measures from the signal measures of the nociception data segments. Next, different orders of power are calculated for each of the three signal measures to generate powers of the three signal measures in step 94 of method 86, shown in FIG. 9. In step 96 of method 86, the powers of the three signal measures are then multiplied together to generate the product of the powers of the three signal measures. Step 98 of method 86 is to perform receiver operating characteristic (ROC) analysis of the product of the powers to arrive at a combinatorial measure for the three signal measures. Steps 92, 94, 96, and 98 are repeated until all of the combinatorial measures have been computed between all of the signal measures of nociception data segments 66.

The signal measures with the most predictive top combinatorial measures (i.e., combinatorial measures satisfying a threshold prediction criteria) are selected, in step 100 of method 86, to perform machine learning. The signal measures with the most predictive top combinatorial measures are top signal measures for nociception data segments 66 and are labeled as the nociception detection input features. With the nociception detection input features determined, hemodynamic monitor 10 is trained or programmed to perform waveform analysis on the arterial pressure waveform of patient 36 (shown in FIG. 4) and extract the nociception detection input features from the arterial pressure waveform of patient 36.

A change detection algorithm, such as a cumulative sum (CUSUM) algorithm can be used to refine model outputs derived from the various input features determined by method 86. More specifically, a CUSUM algorithm can be used to independently determine nociception events and stable episodes in the clinical dataset 60 using arterial pressure waveform heart rate and blood pressure. The CUSUM algorithm can be designed to detect changes beyond a particular threshold (e.g., 10 mmHg) in a signal measure (e.g., blood pressure) over time. Other signal measures, such as contractility and heart rate variability, could alternatively be used as the at least two signal measures. Simultaneous changes in both heart rate and blood pressure, as detected by the CUSUM algorithm, can be a reliable indication of nociception or non-nociception in patient 36, as is discussed in greater detail below.

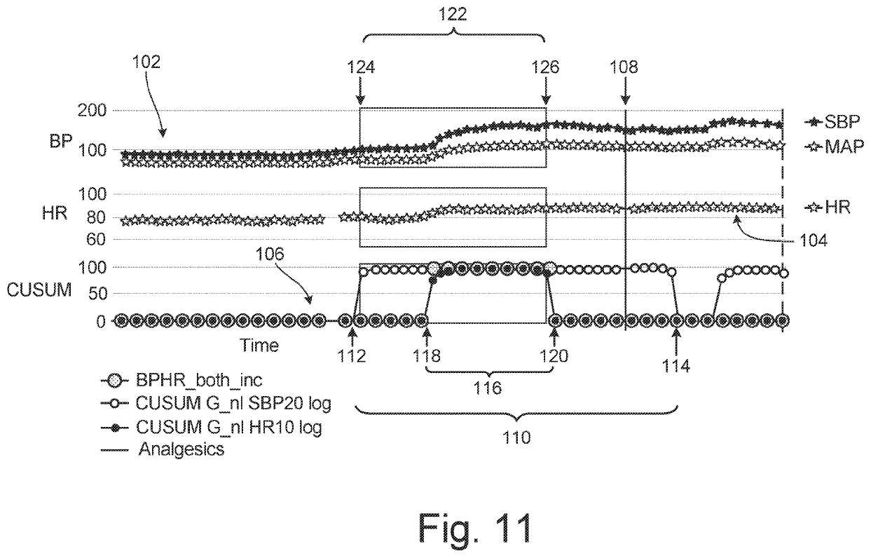

FIG. 11 is a graph illustrating systolic blood pressure 102 from clinical dataset 60, heart rate 104 from clinical dataset 60, and output 106 of the CUSUM algorithm for each of systolic blood pressure 102 and heart rate 104 all plotted over time. The particular period of time can range from 1 minute to 15 minutes in an exemplary embodiment, but longer periods are contemplated herein. Analgesic label 108 is also included to show that the clinical patient of this dataset was administered an analgesic at the time position of analgesic label 108. Output 106 includes systolic blood pressure event (hereinafter referred to as “SBP event”) 110 which is a change, and more specifically, an increase in systolic blood pressure as determined by the CUSUM algorithm applied to the plot of systolic blood pressure 102. SBP event 110 is defined by starting point 112 and ending point 114. Output 106 also includes heart rate event (hereinafter “HR event”) 116 which is a change, and more specifically, an increase in heart rate as determined by the CUSUM algorithm applied to the plot of heart rate 104. HR event 116 is defined by starting point 118 and ending point 120.

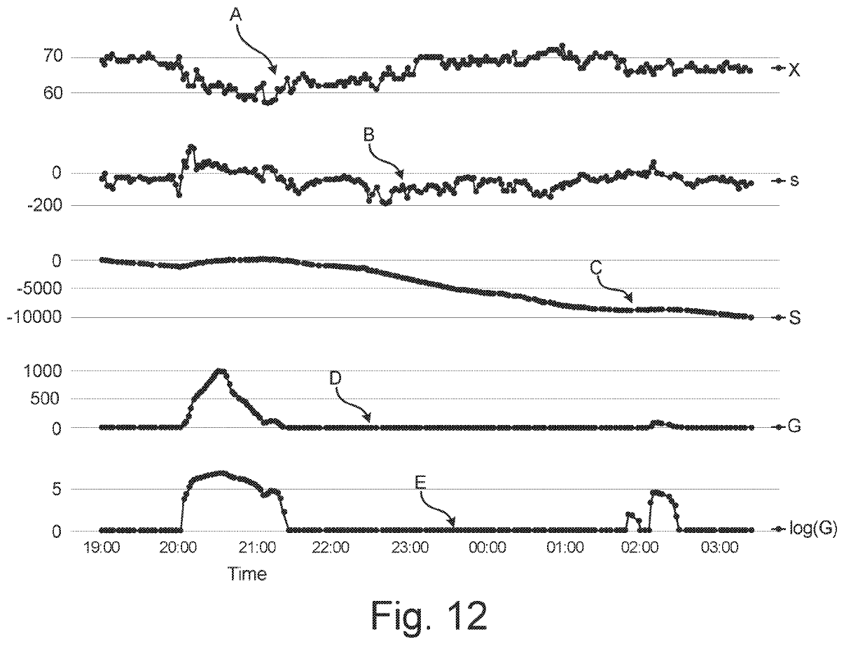

In general, any overlap between SBP event 110 and HR event 116 represents a simultaneous change (i.e., simultaneous increase) indicative of nociception. Where simultaneous change is detected, the earlier starting point of either SBP event 110 or HR event 116 is considered to be the starting point of nociception event 122. The earlier ending point of either SBP event 110 or HR event 116 is considered to be the ending point of nociception event 122. As shown in FIG. 11, starting point 112 of SBP event 110 corresponds with starting point 124 of nociception event 122, and ending point 120 of HR event 116 corresponds with ending point 126 of nociception event 122. FIG. 12, which is described below, provides an example of how the CUSUM algorithm can be applied to the plot of systolic blood pressure 102 or the plot of heart rate 104 to generate the output 106 of the CUSUM algorithm.

FIG. 12 shows a series of graphs with plots A through E demonstrating how the CUSUM algorithm can be applied to the plot of systolic blood pressure 102, the plot of heart rate 104, and/or the plot of any other signal measure that can be used to determine nociception of patient 36. Plot A in FIG. 12 is a plot of a signal measure X over time. The signal measure X can be systolic blood pressure 102, heart rate 104, and/or any other signal measure that can be used to determine nociception of patient 36.

The CUSUM algorithm can use an instantaneous change value s to calculate the cumulative sum for the plot A of signal measure X. The instantaneous change value s can be represented by plot B in FIG. 12 and the equation below:

$${s}_{i} = \frac{Δ}{{σ}_{i - t}} * [ ( {X}_{i} - {X}_{i - t} ) - ( \frac{Δ}{2} ) ] \qquad ( Equation 1 )$$

where i is a current time point and t is how far back in time in the plot A of signal measure X the CUSUM algorithm goes to make a comparison. For example, t can be fifteen minutes. In Equation 1, X is a value of the signal measure X plot at an indicated time, Δ is a predetermined change value (such as 10 mmHg delta for systolic blood pressure or a ten-percent change decrease), σ is a standard deviation of X, and s is an instantaneous change value for the plot A of signal measure X. The instantaneous change value s will only be positive when the difference between Xᵢ−Xᵢ₋ₜ in Equation 1 is greater than Δ/2 in Equation 1. The instantaneous change value s is used by the CUSUM algorithm to calculate a cumulative sum S of the plot A of signal measure X, which is represented by plot C in FIG. 12 and by the equation below:

$${S}_{i} = {S}_{i - 1} + {s}_{i} \qquad ( Equation 2 )$$

The cumulative sum S shown in plot C and determined by Equation 2 is used to calculate change signal G of signal measure X, which is represented by plot D in FIG. 12 and the equation below:

$${G}_{i} = {S}_{i} - min [ {S}_{i - t} : {S}_{i} ] \qquad ( Equation 3 )$$

where i is the current time point from Equation 1 and t is how far back in time the CUSUM algorithm goes to make a comparison in Equation 1. The change signal Gᵢ in Equation 3 only becomes positive if the value of the cumulative sum Sᵢ increases in time. Also, change signal Gᵢ becomes larger as the difference between Xᵢ−Xᵢ₋ₜ in Equation 1 increases. If the value of the cumulative sum Sᵢ decreases or remains constant with time, the change signal Gᵢ will be zero. Before outputting plot D of the change signal Gᵢ to display 12 of hemodynamic monitor 10, the CUSUM algorithm can take the logarithm of the change signal Gᵢ to further accentuate the positive values of the change signal Gᵢ. Plot E in FIG. 12 is the logarithm of change signal Gᵢ from plot D. Plot E can be outputted by hemodynamic monitor 10 to display 12 for each signal measure being used to identify nociception events 122 by hemodynamic monitor 10 (such as systolic blood pressure 102 and heart rate 104).

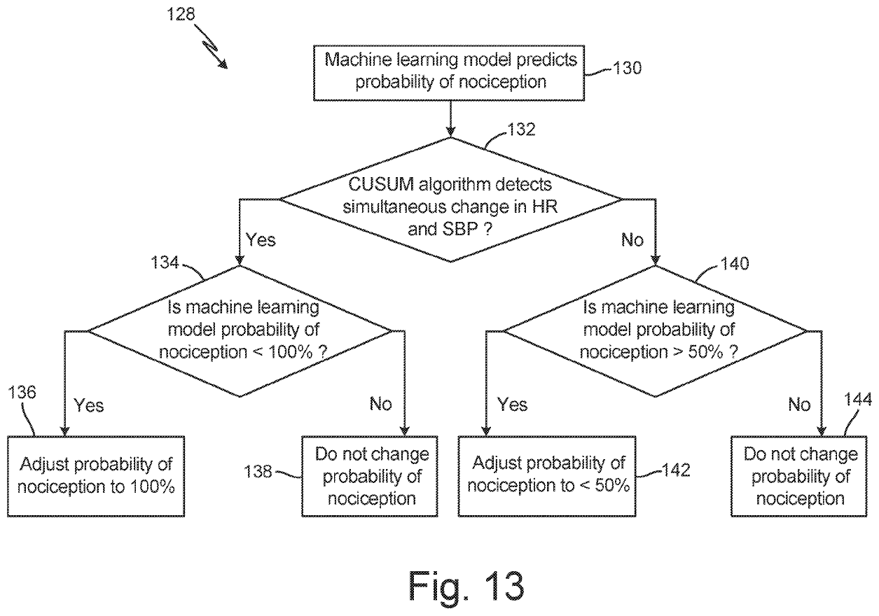

The CUSUM algorithm can additionally and/or alternatively be used to identify stable episodes where no significant changes in systolic blood pressure or heart rate are detected. For example, a stable episode plotted by the CUSUM algorithm would have an output 106 where the output plot for systolic blood pressure 102 can be a flat line with a zero value and the output plot for heart rate 104 can be a flat line with a zero value. Such an episode can be represented in plots substantially similar to those shown in FIG. 7 indicating a stable period with no nociception. As was the case with stable data segment 72 of FIG. 7, periods with no nociception, as detected by the CUSUM algorithm can be labeled with starting and ending points (i.e., starting point 74 and ending point 76), and the period of time defined by the starting and ending points labeled as a stable data segment (i.e., stable data segment 72). The classification-type machine learning model of hemodynamic monitor 10 can be trained using nociception data segments 66, labeled positive, and stable data segments 72, labeled negative, to output a probability of nociception versus a stable episode. As discussed below with reference to FIG. 13, nociception events 122 and stable episodes identified by the CUSUM algorithm in the clinical dataset 60 can be compared with the nociception data segments 66 and stable data segments 72 determined by the machine learning model described above with reference to FIGS. 5-10.

FIG. 13 is a flowchart illustrating select steps of method 128 for using the CUSUM algorithm to refine the machine learning model described above with reference to FIGS. 5-10. More specifically, changes in systolic blood pressure and heart rate detected by the CUSUM algorithm (FIG. 11) are used to increase or decrease probability risk scores from the machine learning model. The machine learning model predicts the probability of a nociception event in step 130. The probability is a function of the various model input features as determined using method 86.

Method 128 proceeds to step 132 to determine if the CUSUM algorithm detects a simultaneous change in systolic blood pressure and heart rate, as illustrated in FIG. 11. If a simultaneous change is detected (i.e., nociception event 122), method 128 proceeds to step 134 to determine if the machine learning model probability is less than 100%. If the probability is less than 100%, method 128 proceeds to step 136 where the probability is adjusted upward to be 100%. This reflects the confidence of the simultaneous change detected by the CUSUM algorithm and corrects the machine learning model from predicting a non-nociception event when the CUSUM algorithm is predicting a nociception event. If the probability is not less than 100% at step 134, method 128 instead proceeds to step 138 where the machine learning model's probability is not adjusted.

Returning to step 132, if the CUSUM algorithm does not detect a simultaneous change in systolic blood pressure and heart rate, method 128 proceeds to step 140 to determine if the machine learning model probability is greater than 50%. If the probability is greater than 50%, method 128 proceeds to step 142 where the probability is adjusted downward to less than 50%. Similar to step 136, this adjustment in the model probability reflects the confidence of the lack of detection of a simultaneous change by the CUSUM algorithm and corrects the machine learning model from predicting a non-nociception event when the CUSUM algorithm is predicting a nociception event. If the probability is not greater than 50% at step 140, method 128 instead proceeds to step 144 where the machine learning model's probability is not adjusted. The probability determined from any of steps 136, 138, 142, and 144 can be displayed by hemodynamic monitor 10. It should be understood that probability maximum and minimum values other than 100% and 50%, respectively, (e.g., 85% and 35%) can be used without departing from the scope of the invention.

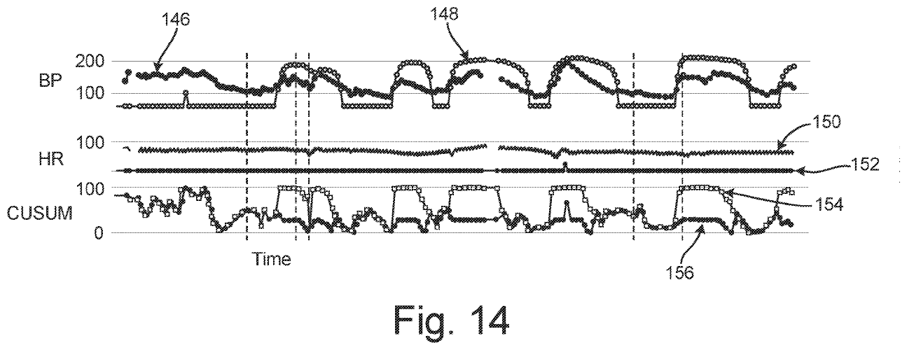

FIG. 14 is a graph illustrating systolic blood pressure 146 from clinical dataset 60 along with a plot of CUSUM systolic blood pressure 148 superimposed on the same graph. FIG. 14 also includes a graph for heart rate 150 from clinical dataset 60 along with a plot of CUSUM heart rate 152 superimposed on the same graph as heart rate 150. The bottom graph in FIG. 14 shows an output of the machine learning model described in FIGS. 5-10 with machine learning model nociception prediction (hereinafter “ML nociception prediction”) 154. The bottom graph also shows adjustments 156 made by the CUSUM algorithm to the output of the machine learning model. As can be seen in the CUSUM systolic blood pressure 148 plot of the top graph, there are several instances where systolic blood pressure 146 increases. However, as shown in the CUSUM heart rate 152 plot of the middle graph, there no instances where heart rate 150 significantly increases. Thus, based on the CUSUM systolic blood pressure 148 and CUSUM heart rate 152, the CUSUM algorithm determines that there are no simultaneous changes and no nociception events in the data segment of FIG. 14. Since the CUSUM algorithm predicts there are no nociception events in the data segment, the CUSUM algorithm enacts adjustments 156 on the output of the machine learning model which forces the output of the machine learning model to be lower (e.g., below 50%) than originally calculated by the machine learning model in several instances and eliminates ML nociception predictions 154 in this data segment.

### Discussion of Possible Embodiments

The following are non-exclusive descriptions of possible embodiments of the present invention.

A method for monitoring arterial pressure of a patient and identifying nociception of the patient includes receiving, by a hemodynamic monitor, sensed hemodynamic data representative of an arterial pressure waveform of the patient; performing, by the hemodynamic monitor, waveform analysis of the sensed hemodynamic data to calculate a plurality of signal measures of the sensed hemodynamic data; extracting input features for the hemodynamic monitor from the plurality of signal measures that are indicative of a nociception event of the patient and a stable episode of the patient; monitoring, by a change detection algorithm of the hemodynamic monitor, at least two signal measures of the plurality of signal measures for a simultaneous change in the at least two signal measures; determining, by the hemodynamic monitor based on the input features, a nociception score representing a probability of the nociception event of the patient; adjusting or maintaining, by the hemodynamic monitor, the nociception score based on an output of the change detection algorithm; and displaying, by the hemodynamic monitor, an adjusted nociception score.

The method of the preceding paragraph can optionally include, additionally and/or alternatively, any one or more of the following features, configurations and/or additional components:

Determining, by the hemodynamic monitor based on an absence of a simultaneous change in the plurality of hemodynamic parameters, a stable score representing a probability of a stable episode where the patient is not experiencing a nociception event.

Training the hemodynamic monitor for determining the probability of the stable episode of the patient, wherein training the hemodynamic monitor for determining the probability of the stable episode comprises: identifying, using the change detection algorithm, stable data segments in the clinical dataset, wherein the stable data segments each comprise: stable blood pressure with no increase greater than a first threshold amount over a set period of time; and stable heart rate with no increase greater than a second threshold amount over the set period of time; identifying a starting point and an ending point of the stable blood pressure and the stable heart rate; labeling the stable data segments from the starting point to the ending point of the stable blood pressure and the stable heart rate; performing waveform analysis of the labeled stable data segments to calculate a plurality of stable signal measures of the stable data segments; and determining at least a portion of the input features by computing combinatorial measures between the plurality of stable signal measures and selecting stable signal measures from the plurality of stable signal measures with most predictive combinatorial measures as belonging to the input features.

Training the hemodynamic monitor for determining the probability of the current nociception event of the patient, wherein training the hemodynamic monitor comprises: collecting a clinical dataset including arterial pressure waveforms and clinical annotations of administration of a compound that alters cardiovascular hemodynamics; identifying, using the change detection algorithm, nociception data segments in the clinical dataset, wherein the nociception data segments each comprise: an increase in blood pressure of at least the first threshold amount compared to a prior time period; an increase in heart rate of at least the second threshold amount compared to the prior time period; and no infusion of the compound that alters cardiovascular hemodynamics started prior to the increase in blood pressure and the increase in heart rate; identifying a starting point and an ending point of the increase in the blood pressure and the increase in the heart rate; labeling the nociception data segments after the starting point and during the increase in the blood pressure and the increase in the heart rate; performing waveform analysis of the labeled nociception data segments to calculate a plurality of signal measures of the nociception data segments; and determining at least a portion of the input features by computing combinatorial measures between the plurality of signal measures and selecting signal measures from the plurality of signal measures with most predictive combinatorial measures as belonging to the input features.

The change detection algorithm comprises: processing, by the hemodynamic monitor, both the blood pressure and the heart rate through a cumulative sum (CUSUM) algorithm to acquire a first CUSUM output for the blood pressure and a second CUSUM output for the heart rate; monitoring, by the hemodynamic monitor, the first CUSUM output for an increase above the first threshold; monitoring, by the hemodynamic monitor, the second CUSUM output for an increase above the second threshold; and detecting a nociception data segment when the increase in the first CUSUM output over the first threshold overlaps in time with the increase in the second CUSUM output over the second threshold.

Adjusting the nociception score based on an output of the change detection algorithm comprises: identifying, using the change detection algorithm, an increase in systolic blood pressure in a time period; identifying, using the change detection algorithm, an increase in heart rate in the time period; and labeling an overlap in the increase in systolic blood pressure and heart rate as a simultaneous change indicative of a nociception event.

Adjusting the nociception score based on an output of the change detection algorithm further comprises adjusting the nociception score to a maximum value if the nociception score is below the maximum value and a simultaneous change is detected by the change detection algorithm.

Adjusting the nociception score based on an output of the change detection means further comprises adjusting the nociception score to a minimum value if the nociception score is above the minimum value and a simultaneous change is not detected by the change detection algorithm.

The at least two signal measures comprise blood pressure and heart rate.

A system for monitoring arterial pressure of a patient and providing a warning to medical personnel of nociception of the patient, the system comprising: a hemodynamic sensor that produces hemodynamic data representative of an arterial pressure waveform of the patient; a system memory that stores nociception detection software code; a user interface that includes a sensory alarm that provides a sensory signal to warn the medical personnel of a nociception event of the patient; and a hardware processor that is configured to execute the nociception detection software code to: perform waveform analysis of the hemodynamic data to determine a plurality of signal measures; extract detection input features from the plurality of signal measures that are indicative of the nociception event of the patient; monitor at least two signal measures from the plurality of signal measures for a simultaneous change in the at least two signal measures; determine, based on the detection input features and a presence of the simultaneous change in the at least two signal measures, a nociception score representing a probability of the nociception event of the patient; and invoke the sensory alarm of the user interface in response to the nociception score satisfying a predetermined detection criterion.

The system of the preceding paragraph can optionally include, additionally and/or alternatively, any one or more of the following features, configurations and/or additional components:

A change detection algorithm monitors the plurality of hemodynamic parameters for a simultaneous change in the plurality of hemodynamic parameters.

The detection input features of the nociception detection software code are determined by detection machine training, wherein the detection machine training comprises: collecting a clinical dataset containing arterial pressure waveforms and clinical annotations of administrations of a compound that alters cardiovascular hemodynamics; identifying nociception data segments in the clinical dataset, wherein the nociception data segments each comprise: an increase in blood pressure of at least a first threshold amount compared to a prior time period; an increase in heart rate of at least a second threshold amount compared to the prior time period; and no infusion of the compound that alters cardiovascular hemodynamics started prior to the increase in blood pressure and the increase in heart rate; identifying a starting point and an ending point of the increase in the blood pressure and the increase in the heart rate; labeling the nociception data segments after the starting point and during the increase in the blood pressure and the increase in the heart rate; performing waveform analysis of the labeled nociception data segments to calculate a plurality of signal measures of the nociception data segments; and determining the detection input features by computing combinatorial measures between the plurality of signal measures of the nociception data segments and selecting top signal measures from the plurality of signal measures of the nociception data segments with most predictive combinatorial measures and labeling the top signal measures as the detection input features.

The system memory stores stable detection software code for determining a probability of a stable episode of the patient, the hardware processor being configured to execute the stable detection software code to: extract stable detection input features from the plurality of signal measures that are indicative of the stable episode of the patient; and determine, based on the stable detection input features, a stable score representing a probability of the stable episode where the patient is not experiencing a nociception event.

The stable software detection code is further configured to determine the stable score based on an absence of a simultaneous change in the plurality of hemodynamic parameters, as determined by the change detection algorithm.

The stable detection input features of the stable detection software code are determined by stable detection machine training, wherein the stable machine training comprises: identifying stable data segments in the clinical dataset, wherein the stable data segments each comprise: stable blood pressure with no increase greater than the first threshold amount over a set period of time; stable heart rate with no increase greater than the second threshold amount over the set period of time; and no infusion performed of a compound that alters cardiovascular hemodynamics; identifying a starting point and an ending point of the stable blood pressure and the stable heart rate; labeling the stable data segments from the starting point to the ending point of the stable blood pressure and the stable heart rate; performing waveform analysis of the labeled stable data segments to calculate a plurality of stable signal measures of the stable data segments; and determining the stable detection input features by computing combinatorial measures between the plurality of stable signal measures and selecting stable signal measures from the plurality of stable signal measures with most predictive combinatorial measures as being the stable detection input features.

Performing waveform analysis of the labeled nociception data segments to calculate a plurality of signal measures of the nociception data segments comprises: identifying individual cardiac cycles in the arterial pressure waveform of the clinical dataset; identifying a dicrotic notch in each of the individual cardiac cycles; identifying a systolic rise phase, a systolic decay phase, and a diastolic phase in each of the individual cardiac cycles; and extracting signal measures from each of the systolic rise phase, the systolic decay phase, and the diastolic phase from each of the individual cardiac cycles.

The signal measures correspond to hemodynamic effects from each of the systolic rise phase, the systolic decay phase, and the diastolic phase from each of the individual cardiac cycles, and wherein the hemodynamic effects comprise contractility, aortic compliance, stroke volume, vascular tone, afterload, and full cardiac cycle.

The signal measures comprise a mean, a maximum, a minimum, a duration, an area, a standard deviation, derivatives, and/or morphological measures from each of the systolic rise phase, the systolic decay phase, and the diastolic phase from each of the individual cardiac cycles.

The signal measures comprise heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles.

The at least two signal measures comprise blood pressure and heart rate.

Computing the combinatorial measures between the plurality of signal measures of the nociception data segments comprises: performing step one by arbitrarily selecting three signal measures from the plurality of signal measures of the nociception data segments; performing step two by calculating different orders of power for each of the three signal measures to generate powers of the three signal measures; performing step three by multiplying the powers of the three signal measures together to generate the product of the powers of the three signal measures; performing step four by performing receiver operating characteristic (ROC) analysis of the product of the powers to arrive at a combinatorial measure for the three signal measures; and repeating steps one, two, three, and four until all combinatorial measures have been computed between all of the plurality of signal measures of the nociception data segments.

The hemodynamic sensor is a noninvasive hemodynamic sensor that is attachable to an extremity of the patient.

The hemodynamic sensor is a minimally invasive arterial catheter based hemodynamic sensor.

The hemodynamic sensor produces the hemodynamic data as an analog hemodynamic sensor signal representative of the arterial pressure waveform of the patient.

An analog-to-digital converter that converts the analog hemodynamic sensor signal to digital hemodynamic data representative of the arterial pressure waveform of the patient.

A method for monitoring arterial pressure of a patient and identifying nociception of the patient, the method comprising: receiving, by a hemodynamic monitor, sensed hemodynamic data representative of an arterial pressure waveform of the patient; performing, by the hemodynamic monitor, waveform analysis of the sensed hemodynamic data to calculate a first signal measure and a second signal measure of the sensed hemodynamic data; processing, by the hemodynamic monitor, both the first signal measure and the second signal measure through a cumulative sum (CUSUM) algorithm to acquire a first CUSUM output for the first signal measure and a second CUSUM output for the second signal measure; monitoring, by the hemodynamic monitor, the first CUSUM output and the second CUSUM output for a change in both the first CUSUM output and the second CUSUM output; detecting a nociception event of the patient when the change in the first CUSUM output overlaps in time with the change in the second CUSUM output; and outputting to a user interface of the hemodynamic monitor a sensory signal to warn medical personnel of the nociception event of the patient.

The method of the preceding paragraph can optionally include, additionally and/or alternatively, any one or more of the following features, configurations and/or additional components:

Performing waveform analysis of the sensed hemodynamic data to calculate the first signal measure and the second signal measure of the sensed hemodynamic data comprises: identifying individual cardiac cycles in the arterial pressure waveform of the sensed hemodynamic data of the patient; identifying a dicrotic notch in each of the individual cardiac cycles; identifying a systolic rise phase, a systolic decay phase, and a diastolic phase in each of the individual cardiac cycles; and extracting signal measures from each of the systolic rise phase, the systolic decay phase, and the diastolic phase from each of the individual cardiac cycles.

The signal measures correspond to hemodynamic effects from each of the systolic rise phase, the systolic decay phase, and the diastolic phase from each of the individual cardiac cycles, and wherein the hemodynamic effects comprise contractility, aortic compliance, stroke volume, vascular tone, afterload, and full cardiac cycle.

The signal measures comprise a mean, a maximum, a minimum, a duration, an area, a standard deviation, derivatives, and/or morphological measures from each of the systolic rise phase, the systolic decay phase, and the diastolic phase from each of the individual cardiac cycles.

The signal measures comprise heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles.

The first signal measure and the second signal measure are selected from the signal measures.

The first signal measure is systolic pressure and the second signal measure is heart rate.

The change in the first CUSUM output is an increase above a first threshold and the change in the second CUSUM output is an increase above a second threshold.

A system for monitoring of arterial pressure of a patient and providing a warning to medical personnel of nociception of the patient, the system comprising: a hemodynamic sensor that produces hemodynamic data representative of an arterial pressure waveform of the patient; a system memory that stores nociception detection software code; a user interface that includes a sensory alarm that provides a sensory signal to warn the medical personnel of a nociception event of the patient; and a hardware processor that is configured to execute the nociception detection software code to: perform waveform analysis of the hemodynamic data to determine a first signal measure and a second signal measure; process both the first signal measure and the second signal measure through a cumulative sum (CUSUM) algorithm to acquire a first CUSUM output for the first signal measure and a second CUSUM output for the second signal measure; monitor over time the first CUSUM output and the second CUSUM output for a change in both the first CUSUM output and the second CUSUM output; detect a nociception event of the patient when the change in the first CUSUM output overlaps in time the change in the second CUSUM output; and output to a user interface of the hemodynamic monitor a sensory signal to warn medical personnel of the nociception event of the patient.

While the invention has been described with reference to an exemplary embodiment(s), it will be understood by those skilled in the art that various changes may be made and equivalents may be substituted for elements thereof without departing from the scope of the invention. For example, while the CUSUM algorithm can be used to adjust the machine learning model of hemodynamic monitor 10, hemodynamic monitor 10 can also use the CUSUM algorithm directly to detect a nociception event of patient 36 from the sensed hemodynamic data of patient 36. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A method for monitoring arterial pressure of a patient and identifying nociception of the patient, the method comprising:
receiving, by a hemodynamic monitor, sensed hemodynamic data representative of an arterial pressure waveform of the patient;
performing, by the hemodynamic monitor, waveform analysis of the sensed hemodynamic data to calculate a plurality of signal measures of the sensed hemodynamic data;
extracting input features for the hemodynamic monitor from the plurality of signal measures that are indicative of a nociception event of the patient and a stable episode of the patient;
monitoring, by a change detection algorithm of the hemodynamic monitor, at least two signal measures of the plurality of signal measures for a simultaneous change in the at least two signal measures;
determining, by the hemodynamic monitor based on the input features, a nociception score representing a probability of the nociception event of the patient;
adjusting or maintaining, by the hemodynamic monitor, the nociception score based on an output of the change detection algorithm; and
displaying, by the hemodynamic monitor, an adjusted nociception score.

2. The method of claim 1, and further comprising determining, by the hemodynamic monitor based on an absence of a simultaneous change in the plurality of hemodynamic parameters, a stable score representing a probability of a stable episode where the patient is not experiencing a nociception event.

3. The method of claim 2, and further comprising training the hemodynamic monitor for determining the probability of the stable episode of the patient, wherein training the hemodynamic monitor for determining the probability of the stable episode comprises:
identifying, using the change detection algorithm, stable data segments in the clinical dataset, wherein the stable data segments each comprise:
stable blood pressure with no increase greater than a first threshold amount over a set period of time; and
stable heart rate with no increase greater than a second threshold amount over the set period of time;
identifying a starting point and an ending point of the stable blood pressure and the stable heart rate;
labeling the stable data segments from the starting point to the ending point of the stable blood pressure and the stable heart rate;
performing waveform analysis of the labeled stable data segments to calculate a plurality of stable signal measures of the stable data segments; and
determining at least a portion of the input features by computing combinatorial measures between the plurality of stable signal measures and selecting stable signal measures from the plurality of stable signal measures with most predictive combinatorial measures as belonging to the input features.

4. The method of claim 3, and further comprising training the hemodynamic monitor for determining the probability of the current nociception event of the patient, wherein training the hemodynamic monitor comprises:
collecting a clinical dataset including arterial pressure waveforms and clinical annotations of administration of a compound that alters cardiovascular hemodynamics;
identifying, using the change detection algorithm, nociception data segments in the clinical dataset, wherein the nociception data segments each comprise:
an increase in blood pressure of at least the first threshold amount compared to a prior time period;
an increase in heart rate of at least the second threshold amount compared to the prior time period; and
no infusion of the compound that alters cardiovascular hemodynamics started prior to the increase in blood pressure and the increase in heart rate;
identifying a starting point and an ending point of the increase in the blood pressure and the increase in the heart rate;
labeling the nociception data segments after the starting point and during the increase in the blood pressure and the increase in the heart rate;
performing waveform analysis of the labeled nociception data segments to calculate a plurality of signal measures of the nociception data segments; and
determining at least a portion of the input features by computing combinatorial measures between the plurality of signal measures and selecting signal measures from the plurality of signal measures with most predictive combinatorial measures as belonging to the input features.

5. The method of claim 4, wherein the change detection algorithm comprises:
processing, by the hemodynamic monitor, both the blood pressure and the heart rate through a cumulative sum (CUSUM) algorithm to acquire a first CUSUM output for the blood pressure and a second CUSUM output for the heart rate;
monitoring, by the hemodynamic monitor, the first CUSUM output for an increase above the first threshold;
monitoring, by the hemodynamic monitor, the second CUSUM output for an increase above the second threshold; and
detecting a nociception data segment when the increase in the first CUSUM output over the first threshold overlaps in time with the increase in the second CUSUM output over the second threshold.

6. The method of claim 5, wherein adjusting the nociception score based on an output of the change detection algorithm comprises:
identifying, using the change detection algorithm, an increase in systolic blood pressure in a time period;
identifying, using the change detection algorithm, an increase in heart rate in the time period;
labeling an overlap in the increase in systolic blood pressure and heart rate as a simultaneous change indicative of a nociception event;
adjusting the nociception score to a maximum value if the nociception score is below the maximum value and a simultaneous change is detected by the change detection algorithm; and
adjusting the nociception score to a minimum value if the nociception score is above the minimum value and a simultaneous change is not detected by the change detection algorithm.

7. A system for monitoring arterial pressure of a patient and providing a warning to medical personnel of nociception of the patient, the system comprising:
a hemodynamic sensor that produces hemodynamic data representative of an arterial pressure waveform of the patient;
a system memory that stores nociception detection software code;
a user interface that includes a sensory alarm that provides a sensory signal to warn the medical personnel of a nociception event of the patient; and
a hardware processor that is configured to execute the nociception detection software code to:
perform waveform analysis of the hemodynamic data to determine a plurality of signal measures;
extract detection input features from the plurality of signal measures that are indicative of the nociception event of the patient;
monitor at least two signal measures from the plurality of signal measures for a simultaneous change in the at least two signal measures using a change detection algorithm;
determine, based on the detection input features and a presence of the simultaneous change in the at least two signal measures, a nociception score representing a probability of the nociception event of the patient; and
invoke the sensory alarm of the user interface in response to the nociception score satisfying a predetermined detection criterion.

8. The system of claim 7, wherein the detection input features of the nociception detection software code are determined by detection machine training, wherein the detection machine training comprises:
collecting a clinical dataset containing arterial pressure waveforms and clinical annotations of administrations of a compound that alters cardiovascular hemodynamics;
identifying nociception data segments in the clinical dataset, wherein the nociception data segments each comprise:
an increase in blood pressure of at least a first threshold amount compared to a prior time period;
an increase in heart rate of at least a second threshold amount compared to the prior time period; and
no infusion of the compound that alters cardiovascular hemodynamics started prior to the increase in blood pressure and the increase in heart rate;
identifying a starting point and an ending point of the increase in the blood pressure and the increase in the heart rate;
labeling the nociception data segments after the starting point and during the increase in the blood pressure and the increase in the heart rate;
performing waveform analysis of the labeled nociception data segments to calculate a plurality of signal measures of the nociception data segments; and
determining the detection input features by computing combinatorial measures between the plurality of signal measures of the nociception data segments and selecting top signal measures from the plurality of signal measures of the nociception data segments with most predictive combinatorial measures and labeling the top signal measures as the detection input features.

9. The system of claim 8, wherein the system memory stores stable detection software code for determining a probability of a stable episode of the patient, the hardware processor being configured to execute the stable detection software code to:
extract stable detection input features from the plurality of signal measures that are indicative of the stable episode of the patient; and
determine, based on the stable detection input features or an absence of a simultaneous change in blood pressure and the heart rate, a stable score representing a probability of the stable episode where the patient is not experiencing a nociception event.

10. The system of claim 9, wherein the stable detection input features of the stable detection software code are determined by stable detection machine training, wherein the stable machine training comprises:
identifying stable data segments in the clinical dataset, wherein the stable data segments each comprise:
stable blood pressure with no increase greater than the first threshold amount over a set period of time;
stable heart rate with no increase greater than the second threshold amount over the set period of time; and
no infusion performed of a compound that alters cardiovascular hemodynamics;
identifying a starting point and an ending point of the stable blood pressure and the stable heart rate;
labeling the stable data segments from the starting point to the ending point of the stable blood pressure and the stable heart rate;
performing waveform analysis of the labeled stable data segments to calculate a plurality of stable signal measures of the stable data segments; and
determining the stable detection input features by computing combinatorial measures between the plurality of stable signal measures and selecting stable signal measures from the plurality of stable signal measures with most predictive combinatorial measures as being the stable detection input features.

11. The system of claim 10, wherein performing waveform analysis of the labeled nociception data segments to calculate a plurality of signal measures of the nociception data segments comprises:
identifying individual cardiac cycles in the arterial pressure waveform of the clinical dataset;
identifying a dicrotic notch in each of the individual cardiac cycles;
identifying a systolic rise phase, a systolic decay phase, and a diastolic phase in each of the individual cardiac cycles; and
extracting signal measures from each of the systolic rise phase, the systolic decay phase, and the diastolic phase from each of the individual cardiac cycles;
wherein the signal measures correspond to hemodynamic effects from each of the systolic rise phase, the systolic decay phase, and the diastolic phase from each of the individual cardiac cycles, and wherein the hemodynamic effects comprise contractility, aortic compliance, stroke volume, vascular tone, afterload, and full cardiac cycle; and wherein the signal measures comprise a mean, a maximum, a minimum, a duration, an area, a standard deviation, derivatives, and/or morphological measures from each of the systolic rise phase, the systolic decay phase, and the diastolic phase from each of the individual cardiac cycles and/or heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles.

12. The system of claim 11, wherein the at least two signal measures comprise blood pressure and heart rate.

13. The system of claim 12, wherein computing the combinatorial measures between the plurality of signal measures of the nociception data segments comprises:
performing step one by arbitrarily selecting three signal measures from the plurality of signal measures of the nociception data segments;
performing step two by calculating different orders of power for each of the three signal measures to generate powers of the three signal measures;
performing step three by multiplying the powers of the three signal measures together to generate the product of the powers of the three signal measures;
performing step four by performing receiver operating characteristic (ROC) analysis of the product of the powers to arrive at a combinatorial measure for the three signal measures; and
repeating steps one, two, three, and four until all combinatorial measures have been computed between all of the plurality of signal measures of the nociception data segments.

14. The system of claim 7, wherein the hemodynamic sensor is a noninvasive hemodynamic sensor that is attachable to an extremity of the patient or a minimally invasive arterial catheter based hemodynamic sensor.

15. A method for monitoring arterial pressure of a patient and identifying nociception of the patient, the method comprising:
receiving, by a hemodynamic monitor, sensed hemodynamic data representative of an arterial pressure waveform of the patient;
performing, by the hemodynamic monitor, waveform analysis of the sensed hemodynamic data to calculate a first signal measure and a second signal measure of the sensed hemodynamic data;
processing, by the hemodynamic monitor, both the first signal measure and the second signal measure through a cumulative sum (CUSUM) algorithm to acquire a first CUSUM output for the first signal measure and a second CUSUM output for the second signal measure;
monitoring, by the hemodynamic monitor, the first CUSUM output and the second CUSUM output for a change in both the first CUSUM output and the second CUSUM output;
detecting a nociception event of the patient when the change in the first CUSUM output overlaps in time with the change in the second CUSUM output; and
outputting to a user interface of the hemodynamic monitor a sensory signal to warn medical personnel of the nociception event of the patient.

16. The method of claim 15, wherein performing waveform analysis of the sensed hemodynamic data to calculate the first signal measure and the second signal measure of the sensed hemodynamic data comprises:
identifying individual cardiac cycles in the arterial pressure waveform of the sensed hemodynamic data of the patient;
identifying a dicrotic notch in each of the individual cardiac cycles;
identifying a systolic rise phase, a systolic decay phase, and a diastolic phase in each of the individual cardiac cycles; and
extracting signal measures from each of the systolic rise phase, the systolic decay phase, and the diastolic phase from each of the individual cardiac cycles;
wherein the signal measures correspond to hemodynamic effects from each of the systolic rise phase, the systolic decay phase, and the diastolic phase from each of the individual cardiac cycles, and wherein the hemodynamic effects comprise contractility, aortic compliance, stroke volume, vascular tone, afterload, and full cardiac cycle; and
wherein the signal measures comprise a mean, a maximum, a minimum, a duration, an area, a standard deviation, derivatives, and/or morphological measures from each of the systolic rise phase, the systolic decay phase, and the diastolic phase from each of the individual cardiac cycles and/or wherein the signal measures comprise heart rate, respiratory rate, stroke volume, pulse pressure, pulse pressure variation, stroke volume variation, mean arterial pressure (MAP), systolic pressure (SYS), diastolic pressure (DIA), heart rate variability, cardiac output, peripheral resistance, vascular compliance, and/or left-ventricular contractility extracted from each of the individual cardiac cycles.

17. The method of claim 16, wherein the first signal measure and the second signal measure are selected from the signal measures.

18. The method of claim 17, wherein the first signal measure is systolic pressure and the second signal measure is heart rate.

19. The method of claim 15, wherein the change in the first CUSUM output is an increase above a first threshold and the change in the second CUSUM output is an increase above a second threshold.

20. A system for monitoring arterial pressure of a patient and providing a warning to medical personnel of nociception of the patient, the system comprising:
a hemodynamic sensor that produces hemodynamic data representative of an arterial pressure waveform of the patient;
a system memory that stores nociception detection software code;
a user interface that includes a sensory alarm that provides a sensory signal to warn the medical personnel of a nociception event of the patient; and
a hardware processor that is configured to execute the nociception detection software code to:
perform waveform analysis of the hemodynamic data to determine a first signal measure and a second signal measure;
process both the first signal measure and the second signal measure through a cumulative sum (CUSUM) algorithm to acquire a first CUSUM output for the first signal measure and a second CUSUM output for the second signal measure;
monitor over time the first CUSUM output and the second CUSUM output for a change in both the first CUSUM output and the second CUSUM output;
detect a nociception event of the patient when the change in the first CUSUM output overlaps in time with the change in the second CUSUM output; and
output to a user interface of the hemodynamic monitor a sensory signal to warn medical personnel of the nociception event of the patient.
